(19) United States
(12) Patent Application Publication
(10) Pub. No.: US 2010/0063409 A1
(43) Pub. Date: Mar. 11, 2010

(54) INTERACTIVE ALCOMETRY

(76) Inventor: Bertil Hök, Vasteras (SE)

Correspondence Address: STITES & HARBISON PLLC, 1199 NORTH FAIRFAX STREET, SUITE 900, ALEXANDRIA, VA, 22314, US

(21) Appl. No.: 12/524,601

(22) PCT Filed: Jan. 31, 2008
(86) PCT No.: PCT/SE2008/050124

§ 371 Date: Nov. 6, 2009

(30) Foreign Application Priority Data
Feb. 1, 2007 (SE) 0700236-3

(51) Int. Cl. A61B 5/097

(52) U.S. Cl. 600/532

(57) ABSTRACT

The invention relates to method and apparatus for the determination of alcohol concentration of expired air. By visualising current or accumulated measuring value and error in real time during ongoing measurement, interactive control of alcohol determination is enabled with respect to accuracy and time lapse. Preferably the measurement is performed without contact with compensation for the dilution of the breath sample, which is performed by simultaneous measurement of temperature, concentration of water vapour or carbon dioxide. The method is divided into a stepwise procedure based on the dependence of the measuring error on the dilution of the expired air, and accumulated time, both being possible to influence by the test person or operator. Special advantages are being obtained at measurements relative to a concentration limit. The alcohol determination is preferably performed by means of absorption spectroscopy within the infrared wavelength range. The apparatus according to the invention includes sensors for the abovementioned entities, an electronic unit for signal and data processing, a presentation unit, and is embedded in a compact housing for handheld use or integration. The response time of the sensors should not exceed 0.5 seconds in order to fulfil the requirement of real time operation. The housing is controllably openable at measurement, and includes a measuring cell illuminated with collimated infrared radiation and means for active air flow.

The present invention relates to the determination of alcohol concentration in expired air, and addresses central aspects concerning measuring accuracy, response time, and interaction between the measuring object or operator on the one hand, and the measuring apparatus, on the other.

A number of methods and devices for alcometry, i e the determination of alcohol concentration in expired air, are described in the literature. Several of these have rendered widespread use, both as measuring instruments, and as control devices. The alcohol interlock is one example of the latter, preventing vehicle drivers from starting the vehicle without an approved breath sample, i e with an alcohol concentration not exceeding the prevailing concentration limit.

Within alcometry, two measuring principles are dominating, making use of physical and chemical properties of alcohol. The latter category is essentially based on combustion mediated by a catalyst. The alcohol concentration can be determined by measuring the developed combustion energy, for example, in a fuel cell or a semiconductor sensor. This sensor type is advantageous in terms of simplicity in design, and peripherals, such as electronic circuitry. Additionally, the semiconductor sensors have small physical dimensions and can be produced at low cost.

The characteristics of the catalyst, and the actual combustion temperature are determining the selectivity of catalytic sensors. Since other organic substances are also being combusted in a similar manner, absolute specificity is difficult to obtain. Another difficult problem is related to the long term properties of catalyst. Influence from certain substances makes repeated calibrations necessary, and constitutes a risk for manipulation. Such substances, e g gases containing sulphur, are prevailing both in expired air and as air pollutants. Catalytic sensors are unfortunately afflicted with reliability problems which have not been solved in a satisfactory manner. These properties are being used by police for evidential purposes against drunk driving. Measuring low concentrations is, however, demanding in terms of precision, and the IR-based evidential instruments on the market are expensive.

Infrared (IR) spectroscopy represents a physical method of measurement which is not afflicted with the problems mentioned above. This method makes use of the specific “finger print” that gas-phase alcohol produces when illuminated by infrared light. The absorption spectre is due to resonant molecular vibrations, which are specific to the atomic bonds within the molecule. From this the specific properties of the absorption spectre can be deduced, and its associated high selectivity against other substances, and security against manipulation. Furthermore, the use of IR-based instruments requires expert knowledge.

The performance of alcometers is often noted with respect to accuracy over a certain range of measurement. For evidential instruments, accuracy of ±5% is frequently required, whereas ±20% is considered adequate for screening and similar purposes. Alcometers for the consumer market has lower accuracy. Among these and screening instruments, systematic error caused by the poorly controlled condition of the catalyst is dominating. For IR-based instruments, systematic errors can be minimised by a calibration procedure. The remaining error has the character of stochastic noise from the information carrying sensor signal or signals. The error and the noise level can be expressed as a statistic entity, e g the mean square of random variations over a specified bandwidth.

An important aspect of alcometry concerns alcohol determination in relation to recommended or legislated concentration limits, e g for vehicle driving. In Sweden, 0.02% blood alcohol concentration is prevailing as the upper limit for vehicle drivers, corresponding to approximately 0.1 mg/L in a sample of expired air. In a major part of the European Union, the corresponding limit is 0.05% and 0.25 mg/L, respectively.

At measurements referring to a certain concentration limit, the ratio of false positive and negative outputs is a relevant value of performance. High accuracy, i e small error, leads to small probability of false outputs, and vice versa. On the other hand, for this purpose, the accuracy is only interesting in an interval close to the concentration, and rather uninteresting outside this interval.

At police controls and road blocks, sobriety tests are being increasingly performed. The Swedish police force is annually performing close to two million sobriety tests on vehicle drivers, and approximately 15 000 drunk drivers are caught this way. Negative outputs are thus being found in more than 99% of the test occasions. There seems to be a relation between the number of tests performed per unit of time, and the percentage of intoxicated drivers, as well as the number of alcohol related accidents per unit of time. In certain countries the percentage of drunk drivers can be higher than in Sweden, but there is a growing support for sober vehicle driving in many countries. The use of alcohol interlocks have also been successful, not only for conditional withdrawal of driver's license after conviction for drunk driving, but also for quality assurance of transport services.

There are many situations and conditions outside the area of traffic safety, when testing of the sobriety of a person could be motivated. The practice of professions requiring precision, good judgement, dependence on certain individuals could be good reason for sobriety test. Moreover, at events of different kinds, there may be reason for sobriety test, e g at passage control stations.

Unfortunately, many application areas of alcohol determination have been hindered by practical and economic limitations of the present technology and engineering solutions. Besides the already mentioned, there are practical problems related to the actual sampling technique. State of the art solutions require exchangeable and disposable mouthpieces for hygienic reasons. Changing mouthpieces at each measurement is both time consuming, and adds costs. The total time lapse for one test could be several minutes at cold weather, which is unacceptable, e g at passage control, or in a vehicle integrated alcohol interlock.

Summing up, it may be concluded that state of the art alcometry is afflicted with a plurality of problems, touching upon central issues related to accuracy, time lapse, accessibility, reliability, and cost.

The object of the present invention is to solve the problems mentioned above, and related ones. The invention, a novel method and apparatus for the determination of alcohol concentration in expired air, exhibits significant advantages compared to the present state of the art. First, accuracy and time lapse can be adapted to specific requirements at each measuring occasion. Second, considerable cost savings are being achieved, since both cost of material and time lapse for each measurement can be considerably reduced. Third, very high reliability is achieved by avoiding the aging phenomena of e g catalytic sensors. Fourth, the need for maintenance of the apparatus is reduced, since it does not require repeated calibration. This also reduces the cost of maintenance. Fifth, the accessibility of the technique is improved for large groups of users. Sixth, the implementation of the apparatus at low production cost makes its price accessible to larger groups of users.

The characteristics of the method and the apparatus according to the invention are described in the enclosed claims. Below follows a comprehensive description of the basic elements of the method and the apparatus.

Determination of alcohol concentration of breath samples is preferably performed, according to the invention, primarily for hygienic reasons, by sampling without physical contact between the apparatus, and the respiratory organs of the test object. The test object or person is frequently a conscious individual, whose eventual intoxication is the object of investigation. The invention is, however, not limited neither with respect to test objects or detected substance. They are applicable also to higher animal species, and unconscious people, and also to other volatile substances than alcohol.

In a preferred embodiment of the invention, a breath sample is delivered by the test person by blowing towards a sensor arrangement positioned at 10-30 cm distance from the mouth and nose. Consequently, the breath sample is diluted with ambient air, requiring compensation for this dilution in order to make determination of alcohol concentration of the expired air possible. The relation between the externally measured alcohol concentration Cₑₓₜ and the alveolar Cₐₗᵥ, can simply be expressed by equation (1):

Cₑₓₜ=D·Cₐₗᵥ (1)

The variable D expresses the degree of dilution. For undiluted expired air, D=1, and at a very high degree of dilution, D→0.

Equation (2) concerns a relation which makes it possible to determine the degree of dilution D in real time, simultaneously with the corresponding measurement of alcohol concentration is performed, at essentially the same point. It is assumed that there is a measuring entity X which is exposed to the same dilution process as Cₑₓₜ, Cₐₗᵥ. The expression for D is:

$$D = \frac{{X}_{ext} - {X}_{amb}}{{X}_{alv} - {X}_{amb}} \qquad ( 2 )$$

With X_{amb} is meant the measured value taken by the entity X in the ambient of the test object, whereas Xₐₗᵥ is the corresponding measuring value. Examples of entities that can be considered to fulfil the criteria of the assumption are temperature, and concentrations of water vapour and carbon dioxide, respectively.

In the table below typical values are given of the measuring values of these entities in indoor environment:

| | | Water vapour | Carbon dioxide |
|---|---|---|---|
| | Temperature | concentration | concentration |
| | (° C.) | (mg/l) | (% by volume) |
| X_{amb} | 18-28 | 4-20 | <0.1 |
| Xₐₗᵥ | 37 | 44 | 5.3 |

By measurement of Xₑₓₜ and X_{amb}, and insertion of Xₐₗᵥ the value of which could be considered constant, D can be determined. The table indicates that CO₂ concentration has two significant advantages compared to the others. One is that the ambient concentration is very small compared to the alveolar. Thereby the influence from the ambient is at a minimum. Another advantage is that a detected difference in CO₂ concentration is most likely to have alveolar origin. On the other hand, the alveolar CO₂ concentration exhibits a somewhat larger variability than temperature and water vapour concentration. Temperature and humidity measurements have advantages with respect to simplicity, speed and cost. To achieve maximum security it is of course possible to use a combination of more than one measuring entity.

It should be noted that the anatomical and physiological deadspace can also cause dilution of the expired air. The anatomical deadspace includes the upper airways, and is approximately 150 ml for a normal adult person. Expired air from the anatomical deadspace is little mixed with alveolar air. The physiological deadspace is dependent on which substance it refers to, and is influences by e g the solubility of the substance within the mucosa. Taking these differences into account, the reasoning above can also be applied to sampling with a mouthpiece.

Measurement of temperature, concentration of water vapour and carbon dioxide is preferably performed by means of sensors specific to each entity. Temperature can be measured with resistive sensors or thermo elements, both providing adequate accuracy. In order to obtain necessary speed of response, 0.5 seconds or shorter, small physical dimensions and therefore small thermal mass is required. Miniaturised temperature sensors with adequate response time are commercially available. The same holds for the measurement of water vapour concentration and carbon dioxide. The first case deals with a capacitive measuring principle, making use of the high dielectric permittivity of water. For CO₂ determination IR absorption can be used, and thereby be integrated with alcohol determination as described above. CO₂ exhibits specific absorption in a narrow wavelength band around 4.26 μm, whereas alcohol has absorption peaks at 3.4 and 9.5 μm. Water vapour exhibits a relatively broad absorption band at 2.6-2.8 μm.

In order to fulfil the criteria of equation (1) and (2) it is required that the sensors for alcohol and entities X are measured at the same point, setting demands on the size and positioning of the sensors. Alternatively, the air sampling part of the apparatus is supplied with a tubing line, and a pumping device for transport of the breath sample from the actual sampling point to the physical position of the sensors.

By insertion of current values of D and C_{amb} it is easy from equation (1) to determine Cₐₗᵥ. Experiments have shown that it is possible to determine alcohol concentration without physical contact at a distance of up to 30 cm between the mouth/nose of a test person and the sampling point. This assumes, however, the active participation of the test person to blow expired air towards the sensor. Passive measurement without the cooperation of the test person requires a shorter measuring distance, up to 10 cm, since only resting respiration can be assumed.

The inaccuracy IA of the method is determined by the measurement error Cₑᵣᵣₒᵣ in relation to the current measured value Cₐₗᵥ, and can suitably bed described by the ratio between these. Cₑᵣᵣₒᵣ can in its turn be partitioned into relatively constant factors and such that can be influenced from one occasion to another, and even during ongoing measurement. The result of such partitioning is shown in equation (3):

$$IA = \frac{{C}_{error}}{{C}_{alv}} = \frac{{C}_{resolution}}{{C}_{alv} \cdot D \cdot \sqrt{Δ t}} \qquad ( 3 )$$

Cᵣₑₛₒₗᵤₜᵢₒₙ refers to the remaining resolution of the instrument which is difficult to control, and limited by noise sources of fundamental origin, or other random factors. Systematic error sources are assumed to be eliminated by a sufficiently accurate calibration procedure. Cᵣₑₛₒₗᵤₜᵢₒₙ is connotated in the dimension of the measured, in this case alcohol concentration, divided by the square root of the current bandwidth, Δf, √(Hz)⁻¹.

The entities Cₐₗᵥ, and D of equation (3) are assumed to be measured values accumulated during a certain measuring time Δt. Prolongation of this time can therefore be considered identical to a reduction of bandwidth.

Equation (3) illustrates the possibility to influence the inaccuracy either by the degree of dilution D or by the measuring time Δt. In order to increase the accuracy a certain factor from a given starting point, it is either required to increase D by the corresponding factor, or by increasing the measuring time the square of this factor, or a combination of these measures.

In the method according to the invention, the relation of equation (3) is used in each single determination of alcohol concentration to adapt measuring time and accuracy to the object or requirement of that occasion. The adaptation is performed interactively between the measuring apparatus and the user/test person or operator. More specifically, the apparatus leaves in real time, i e without a limiting time delay, indication of current or accumulative measuring value and error or thereto related entity. The factor D can be such entity.

The user, test person or operator can choose at any instant whether to finish the determination, or continue in order to increase accuracy. Such increase is possible according to equation (3) either by prolonging the measuring time, or by changing the expiration of the test person in relation to the apparatus, which brings about an increase of the dilution factor D, and a corresponding increase of accuracy. D is also influenced by the position of the sensors with respect to the test person. An operator can, guided by the indicated accuracy, move the sampling point with respect to the respiratory organs of the test person until acceptable level is obtained. Measurement in real time means that the procedure is possible at the test person's normal respiratory activity, without active cooperation from his/her part. It is therefore possible to perform measurement even in unconscious people, and in animals.

The method according to the invention thus makes possible the interactive control of the accuracy and time lapse of alcohol determination. The relations (1), (2), and (3) are being employed for computation of instant values of Cₐₗᵥ, and D in real time during ongoing measurement, as soon as a significant difference Xₑₓₜ-X_{amb}, occur, apart from normally occurring random variations. A starting criterion for the computation of Cₐₗᵥ, and Cₑᵣᵣₒᵣ is that D is above a certain minimum value, Dₘᵢₙ. The accuracy of the first determination after reaching the threshold Dₘᵢₙ is by definition low, but can rapidly be improved, as higher values of D are obtained.

If the current issue is limited to determine whether the alcohol concentration exceeds a certain concentration limit Cₗᵢₘᵢₜ or not, the method according to the invention is offering particular advantages. If the current measuring value Cₐₗᵥ with addition of the error Cₑᵣᵣₒᵣ is lower than Cₗᵢₘᵢₜ, i e if

Cₐₗᵥ<cₗᵢₘᵢₜ−Cₑᵣᵣₒᵣ (4)

The determination can be finished directly without trying to improve accuracy by prolonged measuring time or adjustment of sensor positions etc. Correspondingly, the same procedure may be adopted when the instant measured value of alcohol concentration exceeds the limit value added to the error:

Cₐₗᵥ>Cₗᵢₘᵢₜ+Cₑᵣᵣₒᵣ (5)

However, if

Cₗᵢₘᵢₜ−Cₑᵣᵣₒᵣ≦Cₐₗᵥ≦Cₗᵢₘᵢₜ+Cₑᵣᵣₒᵣ (6)

then accumulation of instant values should continue in order to reduce the error, with the objective of true knowledge whether the concentration limit is exceeded or not.

The case (4) is valid for more than 99% of randomly selected Swedish vehicle drivers, according to the description above. The method according to the invention should, for this group mean significantly reduced measuring time, and therefore lower cost per sample.

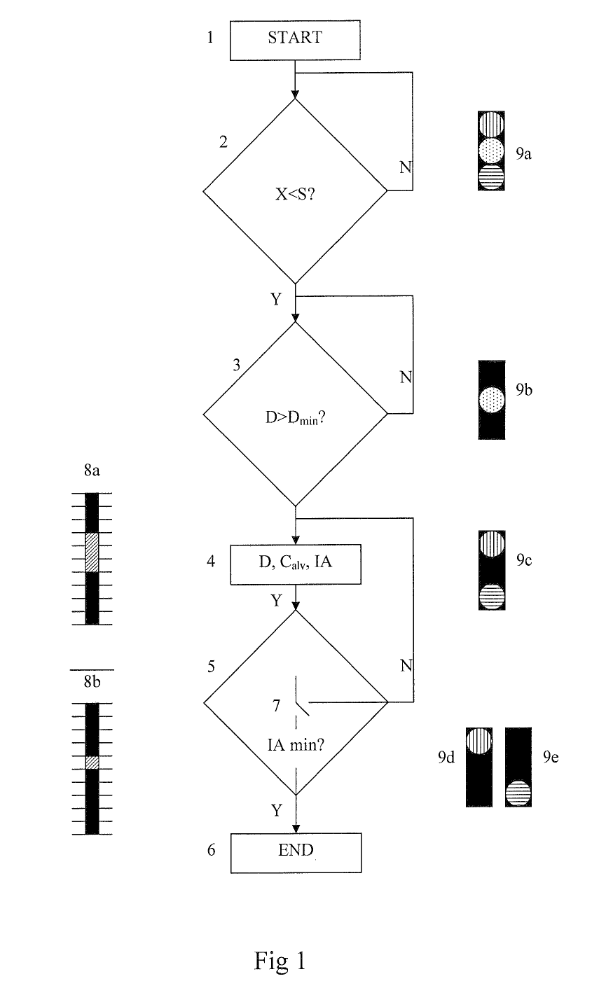

The invention is described in more detail in connection with the enclosed FIGS. 1-4.

FIG. 1 shows a flow chart of the method according to the invention.

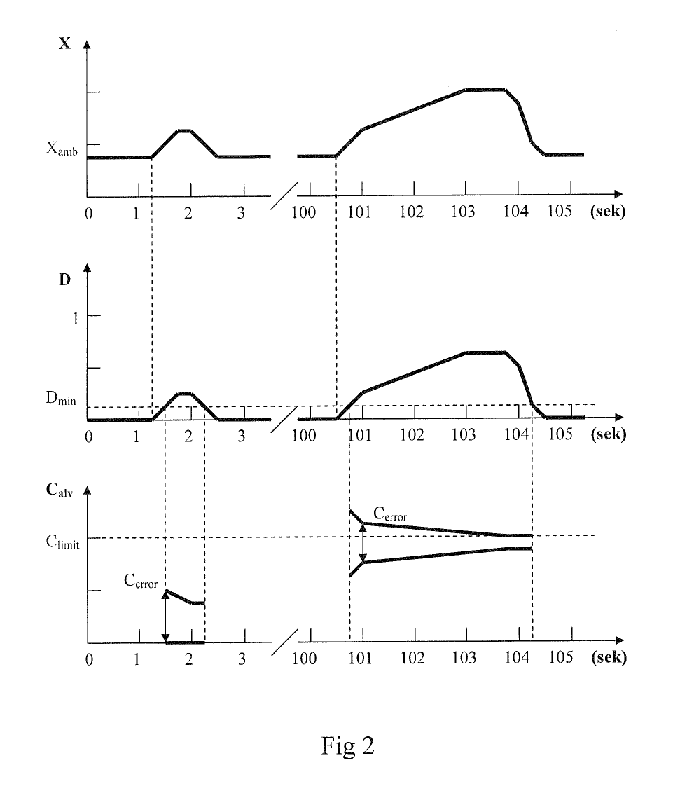

FIG. 2 schematically shows the timing of events during a determination of alcohol according to the invention.

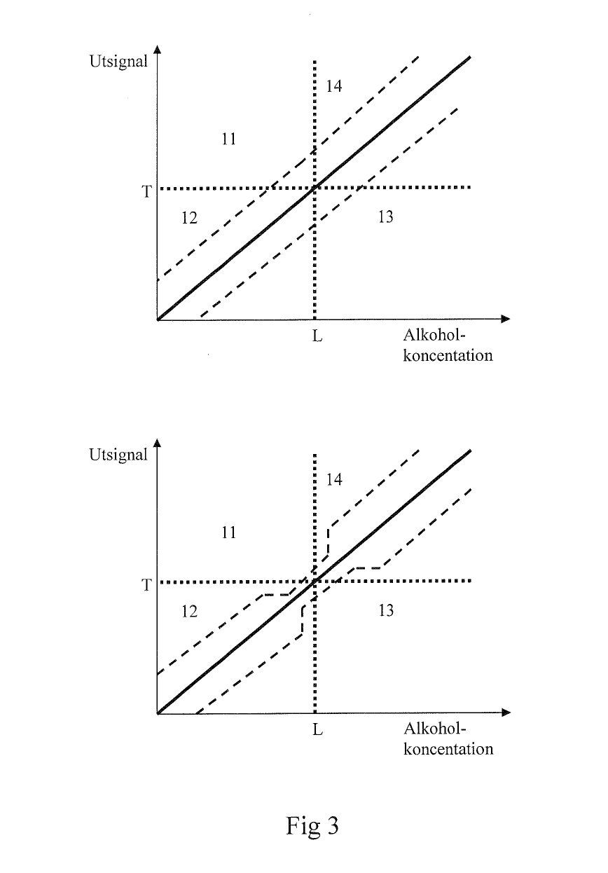

FIG. 3 shows examples of accuracy for different applications.

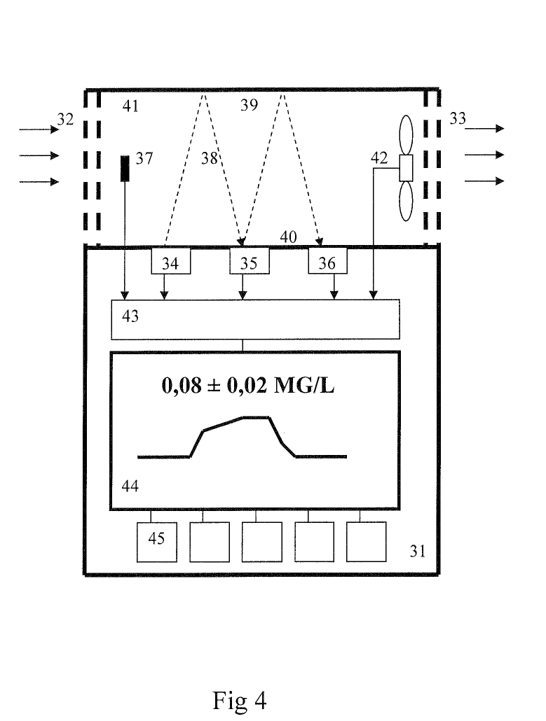

FIG. 4 schematically shows one embodiment of the apparatus according to the invention.

The flow chart of FIG. 1 comprises a number of steps corresponding to the conditions of the apparatus. To start with, at the activation of the apparatus a starting position 1 occurs, and is directly transferred into a waiting mode 2. The activation can be performed manually by an on/off button, or automatically from some other equipment connected to the apparatus. During the waiting mode 2, a stability test is performed of the sensor signals corresponding to alcohol concentration and degree of dilution, respectively. If the sensor signals are being interfered in any way, the waiting process is continuing until stability has been reached.

When the criterion is fulfilled that the variation in time of the abovementioned signals does not exceed a certain threshold S, the apparatus is transferred into the measuring mode 3. In this mode the test person can be instructed to exhale towards the sensors of the apparatus, alternatively, these are positioned without the active cooperation of the test person to the vicinity of his/her respiratory organs. Analysis of the sensor signals corresponding to dilution is being performed continuously, and when the criterion for the dilution factor D>Dₘᵢₙ has been reached, the apparatus is being transferred into a computational mode 4. In this mode, measurement and computing of the entities D, Cₐₗᵥ and IA are being performed simultaneously in real time, and current values of the at least the latter are being communicated, likewise in real time, to the operator, the user or the test person.

During the condition of measuring and computation 4, the operator, user or test person can choose to finish the measurement if sufficient accuracy has already been obtained. Finishing is done by the test person finishing to blow expired air towards the sensors of the apparatus, or by withdrawing these from the vicinity of the respiratory organs. The operator, user or test person can also choose to continue blowing expired air towards the sensors in order to accumulate further measuring points, or eventually increasing the dilution factor D in order to increase accuracy.

The task of carrying out measurements and computations simultaneously in real time sets special demands on the apparatus according to the invention. On the one hand, signals from sensors must be sampled and put in a buffer memory, and from there be transferred to a arithmetic-logic computation unit in which the equations (1), (2), and (3), or corresponding ones, are being executed. The result from the computations, as well as eventual interim results, should also be stored in a buffer memory, and from there be visualised in order to be perceived by the test person or operator.

The time interval between each indication should not exceed 0.5 seconds in order for the test person or operator to perceive the indicated information as instantaneous. With commercially available microprocessors, including both buffer memory and arithmetic-logic unit, it is possible to fulfil this requirement. Preferably, the microprocessor also includes permanent memory for storing the program controlling computations and data communication.

The change in time of the measurement error is being computed and indicated in real time, clearly visible for the test person or operator during the subsequent phase 5. When the accumulated inaccuracy IA reached a lowest value, automatic transfer to a final condition 6 can take place, whereby the accumulated values of Cₐₗᵥ and Cₑᵣᵣₒᵣ, respectively, may be valid as final for the current determination. The instruction of the operator, user or test person concerning the eventual continuation of the measurement or its termination, is however overruling this automation, and may be controlled by a switch position 7 controlled by this person. It is, for example, possible to continue measurement during one or several subsequent expirations.

The various conditions 1-6 are indicated by the apparatus according to the invention by audiovisual or haptic arrangements. For example, the measured value and inaccuracy may be indicated as position and width on a measuring scale 8a, 8b, in which 8a is indicating a relatively large measurement error, whereas 8b indicates essentially the same measuring value but a smaller error.

An alternative embodiment of indication of conditions is shown in the sequence 9a-9e, using symbols of traffic lights. The start- and wait conditions 1 and 2 are indicated in 9a by illumination from red, yellow and green lamps. The measuring condition 3 is indicated by illumination from the yellow lamp 9b, eventually flashing in order to call attention. The condition for measurement and computation 4 is indicated by illumination from both the red and green lamp, 9c. The green and red lamp emits different intensities depending on the current magnitude of the measuring value compared to a concentration limit, using the relations (4), (5), and (6). In the final condition (6) the inaccuracy is in most cases so small that a definite response can be provided, as to whether the concentration limit is exceeded or not. In such case it gives rise to a red stop signal 9d if exceeded, and a green light 9e if not.

FIG. 2 schematically shows typical time sequences of the sensor signal X, and the degree of dilution D deduced from equation (2), and the computed alveolar alcohol concentration, with computed from equations (1) and (3), with accumulated measurement error inserted into the figure as lower and upper limit for the measuring value. The variation as a function of time is shown for the variables X (upper graph), D (middle graph) and Cₐₗᵥ, Cₑᵣᵣₒᵣ (lowest graph) partly for a sober test person (time scale 0-3 sec) and partly for a person with alcohol concentration slightly below the concentration limit Cₗᵢₘᵢₜ (time scale 100-105 sec).

From a stable initial value X_{amb} corresponding to that valid for the ambient, X will continuously rise to a higher value as long as the test person is blowing towards the sensor arrangement. When expired air from the test person has hit the sensor arrangement a signal plateau is reached and maintained as long as the stream of air is maintained, i e at the duration of the expiration. After that the level is decreasing, and retains the initial value X_{amb}.

The variable D varies in time in an identical way as the variable X, which is obvious from equation (2). This is illustrated in the middle curve of FIG. 2. Experiments have shown that D=0.3-0.5 could be a realistic level, obtainable by forced expiration towards a sensor arrangement at a distance of 10-30 cm. This level is, however, obtained after one, or a few seconds of blowing. At an earlier point in time, the abovementioned level Dₘᵢₙ is reached, at which the apparatus switches to the measuring and computational condition. A suitable value could be Dₘᵢₙ=0.1 . . . 0.2. It is first at the point in time when Dₘᵢₙ is exceeded that measured values of alcohol concentration appears which together with the measurement error is illustrated on the lower curve. In the left series of curves, with a sober test person, immediate response that the level is below the concentration limit is provided, and therefore the test person can choose to finish the determination.

In the series of curves at right it is from the beginning not clear whether the concentration limit will be exceeded or not, since this limit falls within the tolerance of the measuring value. The test person therefore chooses to continue the determination, whereby the measurement error decreases. The accumulated measuring error approaches a minimum at the end of the breath.

As illustrated in FIG. 2 the method according to the invention allows for significantly reduced time for measurement and computation when a sober person is subjected to a sobriety test with respect to a given concentration limit.

FIG. 3 shows two diagrams of which the upper concerns a measuring instrument according to the present state of the art, whereas the lower provides an example of the present invention. In both diagrams the output signal (solid lines) and measurement error (dashed lines) are shown as a function of alcohol concentration. Moreover, a concentration limit L is indicated on the axis of alcohol concentration, and a trigger level T on the axis of the output signal. Thereby four quadrants 11, 12, 13, 14 within the diagram are marked. The quadrants 12 and 14 represent truly negative and positive outputs of the apparatus, whereas the quadrants 11 and 13 represent false positive and negative outputs, respectively

In a conventional apparatus the measurement error cannot be influenced by the user, implicating a risk for false outputs according to the upper diagram. According to the present invention, however, it is possible to adapt the measurement error to minimise it at about the concentration limit L and the trigger level T. As already described this adaptation is performed between the user, operator or test person on the one hand, and the apparatus on the other.

FIG. 4 shows schematically the design of the apparatus according to the invention according to a preferred embodiment. The apparatus is preferably embedded in a apparatus box or housing 31, the size of which is adapted to handheld use or integration within vehicle instrumentation, e g a vehicle steering wheel. The physical dimensions of the housing 31 should not exceed 120×120×30 mm. The housing 31 is supplied with openings 32, 33 for in- and outflow of breath sample. Thereby a measuring cell 41 is defined and being trans-illuminated by collimated infrared light from a source 34. The IR beam 38 is reflected a few times against the reflecting surfaces 39, 40 within the measuring cell 41, before it hits the detectors 35, 36 which are adapted to selectively detect radiation within the wavelength bands within which alcohol on the one hand, and water vapour, carbon dioxide on the other exhibits substance specific absorption.

A temperature or flow sensor 37 with response time 0.5 seconds or shorter, is located near the entrance 32 of the measuring cell 41. The sensor 37 can also be combined with an electric heating device in order to heat the incoming breath sample. The purpose may partly be to measure flow velocity according to the principle of hot wire anemometry, and partly to prevent condensation due to the humidity of expired air. Further there is a device 42 for active air flow through the measuring cell 41. This device can for example be a miniature fan or pump.

The openings 32 and 33 to the measuring cell 41 are in FIG. 4 schematically drawn as double gratings for two reasons. Partly the gratings themselves are protecting against dirtying of the sensitive optical elements within the measuring cell 41 and can be supplied with different types of filter for use in particularly harsh environment. Partly double and deflectable gratings which are only opened a short while during measurement provide an effective protection between the measurement occasions.

The electric signals generated by the sensors 35, 36, 37 are subject to amplification, filtering and other signal processing within the electronic unit 43, which also executes control and drive of the IR source 34, the heating device within the sensor 37 and the flow device 42. The electronic unit 43 preferably includes a microprocessor or corresponding, for execution of a program stored in a memory, including the computations and condition indications described above.

The IR source 34 is preferably modulated at a frequency of 2 Hz or higher. Thereby problems with offset drift of the IR detector and input amplifier are eliminated, while obtaining adequate response time. The demands on response time are primarily defined by real time visualisation of measuring values, being perceived as instantaneous by the operator or test person. In practice this corresponds to a response time of 0.5 seconds or less.

The electronic unit 43 preferably includes circuits for digital signal communication to an integrated presentation unit 44 for indication of measuring values and to other units. The presentation unit 44 is in the most simple case a small number of lamps or light emitting diodes as described in conjunction with FIG. 1, but could also be a graphic and/or alphanumeric display as illustrated in FIG. 4, for more detailed indication of the variation in time of the sensor signals. The control of the user or operator on the electronic unit 43 is exercised by manual control devices 45. The supply voltage could either be taken from a built-in battery or from an external voltage source.

The apparatus according to the invention is designed with respect to choice of materials and components, for maintenance-free operation over at least 15 years or 30 000 measuring occasions.

The apparatus according to the invention is designed so that a normally talented person without trouble can use it. The described interaction, including the choice of finishing point for the determination has an intuitive character and therefore demands a minimum of previous instruction.

It should further be noted that the schematically illustrated apparatus of FIG. 4 is built with materials and components which can be manufactured serially at low cost. Moreover the design of the apparatus is adapted to automatic assembly which means that the number of manual fabrication procedures is minimal. Also calibration, test and quality assurance can be effectively performed with automatic means. Conclusively, this implicates that the apparatus according to the invention can be given an attractive price, and thereby reach large groups of users. Elimination of periodic calibration during the life time of the product also contributes to provide the method and apparatus according to the invention low drift cost and high accessibility.

The embodiments of FIG. 1-4 are not as already pointed out not limiting for the applicability of the present invention. Its characteristics are defined by the enclosed claims.

1. Method for the determination of alcohol concentration in expired air, characterised in:
Interaction between apparatus on the one hand and test person or operator on the other
Said interaction referring to adaptation of accuracy and time lapse of said determination to current objective or requirement
Said interaction implying measuring value and error of said determination or thereto related entity being visualised to said test person or operator in real time during ongoing measurement, with possibility of choice for prolonged measuring time and compensation for possible dilution of said expired air, with the purpose of increasing said accuracy
Said visualisation being performed by means of audiovisual or haptic indication from a presentation unit within said apparatus.

2. Method according to claim 1, characterised in that said visualisation having symbolic or numerical character, and being directed towards said test person or operator.

3. Method according to claim 1, characterised in that said measuring error is dependent on the dilution of said expired air, accumulated time of said determination, and noise, interference or other random variations of said measuring value.

4. Method according to claim 1, characterised by contactless sampling of said expired air with compensation for the dilution of the sample by measurement of temperature, concentration of water vapour or carbon dioxide or a combination of these entities.

5. Method according to claim 1, characterised in a stepwise procedure, including a starting condition, at which said apparatus is activated, followed by a waiting condition at which sensor signals corresponding to said measuring value and other values are controlled with respect to stability, subsequently followed by a condition of measuring and computation at which real time alveolar alcohol concentration of alcohol and measuring error is carried out and visualised, and a final condition following from accumulated inaccuracy having reached a minimum.

6. Method according to claim 1, characterised in that said alcohol determination is based on a physical property, e g infrared absorption, and that said measuring error after minimising, essentially has stochastic character.

7. Apparatus for the determination of alcohol concentration according to claim 1 characterised in at least
one first sensor, the output signal of which is a monotonous function of said alcohol concentration
one second sensor, the output signal of which is a monotonous function of the dilution of said expired air, or thereto related entity, whereby the response time of said first and second sensor does not exceed 0.5 seconds
one electronic unit for processing said signals from a program stored in a memory unit, said program intended for control and computations for said determination
one presentation unit for audiovisual or haptic visualisation of said determination, measuring value, or other relevant information.

8. Apparatus according to claim 7, characterised in said apparatus being embedded in housing with outer dimensions not exceeding 120×120×30 mm, including a device for controlled opening of said housing for receiving said expired air.

9. Apparatus according to claim 7, characterised in at least one sensor for the measurement of said temperature or flow velocity of said expired air, whereby the response time of said sensor does not exceed 0.5 seconds.

10. Apparatus according to claim 7, characterised by a measuring cell with an opening for reception of said expired air said measuring cell being trans-illuminated by collimated infrared radiation, and means for active flow therethrough.
